(19) United States
(12) Patent Application Publication
(10) Pub. No.: US 2006/0035342 A1
(43) Pub. Date: Feb. 16, 2006

(54) Engineered enzymes and their use for synthesis of thioglycosides

(76) Inventors: Stephen G Withers, Vancouver (CA); Michael Jahn, Vancouver (CA)

Correspondence Address: OPPEDAHL AND LARSON LLP, P O BOX 5068, DILLON, CO, 80435-5068, US

(21) Appl. No.: 10/527,495

(22) PCT Filed: Sep. 12, 2003
(86) PCT No.: PCT/CA03/01398

§ 371 Date: May 18, 2005

Related U.S. Application Data
(60) Provisional application No. 60/410,502, filed on Sep. 12, 2002.

(51) Int. Cl. C12P 19/60; C07H 21/04; C12N 9/24; C12N 15/74; C12N 1/21; C07H 5/10

(52) U.S. Cl. 435/75; 435/200; 435/252.3; 435/471; 536/23.2; 536/4.1

(57) ABSTRACT

Mutant glycosidases in which the amino acid in the active site that serves as the acid, base or acid/base-catalyst is converted from a carboxylic acid to some other amino acid (for example to a simple alkyl, as in alanine or glycine) can catalyze the reaction of a thiosugar acceptor and an activated donor to form a thioglycoside. The “thioglycoligases” represent a novel class of mutant enzymes, and represent a first aspect of the invention. Thioglycoligases can be used in accordance with the method of the invention to couple a thiosugar acceptor and an activated donor to form a thioglycoside. By selection of the donor and acceptor species, as well as the specific enzyme employed, thioglycosides of different structure and stereochemistry can be obtained.

This application claims the benefit of U.S. Provisional Application No. 60/410,502, filed Sep. 12, 2002, which application is incorporated herein by reference in those jurisdictions permitting such incorporation.

### BACKGROUND OF THE INVENTION

This application relates to engineered enzymes, and to their use for the synthesis of thioglycosides.

Carbohydrate mimetics that are resistant towards enzymatic hydrolysis have proven to be useful as competitive glycosidase inhibitors and therefore have potential as therapeutics. Thioglycosides, in which the glycosidic oxygen has been replaced by sulfur, have been especially valuable as stable glycoside analogues in a range of studies of glycosidases, both as competitive inhibitors, e.g. for α-L-fucosidases,^{[1]} pancreatic α-amylase^{[2}] or for cellulases,^{[3]} and recently in the formation of stable complexes for X-ray crystallography analysis, e.g. with endoglucanase Cel7B from Fusarium oxysporum,^{[4]} maize β-glucosidase ZMGlu1,^{[5]} barley β-D-glucan glucohydrolase,^{[6]} endoglucanase Cel5A^{[7]} or E. coli maltodextrin phosphorylase (MalP),^{[8]} and they are gaining increasing interest as targets for the pharmaceutical industry.^{[9]}

The chemical synthesis of thioglycosides has been achieved via glycosylation of thioacceptors with activated glycosyl donors,^{[10], [11], [12]} via the S_{N}2 reaction of 1-thio sugars with activated acceptors^{[13]} and via Michael addition of 1-thiosugars to α,β-unsaturated systems;^{[14]} all routes involve numerous protection and deprotection steps and require good control of anomeric stereochemistry.

The few naturally occurring thioglycosides belong to the family of glucosinolates and are found in cruciferous plants.^{[15]} The enzymes that catalyze the formation of the thioglycosidic linkages, S-glucosyltransferases, have been purified from plant extracts,^{[16]} as well as cloned into and expressed from E. coli.^{[17]} However, no useful enzymatic syntheses of thioglycosidic linkages in vitro have been reported. Such approaches would be valuable.

The action of retaining glycosidases on glycosides is in most instances mediated by two key active site amino acid residues, the catalytic nucleophile and the catalytic acid/base. We have previously reported on the efficient synthesis of O-glycosidic linkages in oligosaccharides by the use of retaining glycosidases that lack the catalytic nucleophile (glycosynthases), in conjunction with activated donors of the opposite anomeric configuration of the natural substrate.^{[18], [19], [20]}

In order to facilitate the synthesis of thioglycosides of diverse structure, it would be useful to have an enzymatic methodology. It is an object of the present invention to provide such methods for making thioglycosides.

### SUMMARY OF THE INVENTION

It has now been surprisingly found that mutant glycosidases in which the amino acid in the active site that serves as the acid, base or acid/base-catalyst is converted from a carboxylic acid to some other amino acid (for example to a simple alkyl as in alanine or glycine) can catalyze the reaction of a thiosugar acceptor and an activated donor to form a thioglycoside. The “thioglycoligases” represent a novel class of mutant enzymes, and represent a first aspect of the invention. Thioglycoligases can be used in accordance with the method of the invention to couple a thiosugar acceptor and an activated donor to form a thioglycoside. By selection of the donor and acceptor species, as well as the specific enzyme employed, thioglycosides of different structure and stereochemistry can be obtained.

### BRIEF DESCRIPTION OF THE DRAWINGS

FIG. 1 shows the hydrolysis of a disaccharide within the active site of a normal glycosidase enzyme which retains stereochemical configuration during hydrolysis.

FIG. 2 shows the hydrolysis of a disaccharide within the active site of a normal glycosidase enzyme which inverts stereochemical configuration during hydrolysis.

FIG. 3 shows HPLC chromatograms of the Man2A E429A catalyzed reaction of the two competing acceptors pNP-Xyl (5.5 mM) and its thio analogue 6 (5.5 mM) with varying concentrations of DNP-Man: a) 3.75 mM, b) 22 mM. Arrows indicate the elution of the O-linked disaccharides, 10 is the S-linked disaccharide.

FIG. 4 shows the mechanism of a retaining glycosidase: Glycosylation and deglycosylation with wild type A) and acid/base mutant B). DNP=dinitrophenyl, Nu=nucleophile.

FIG. 5 shows enzymatic synthesis of thioglycosides 7 and 9. DNP=dinitrophenyl, pNP=para-nitrophenyl

FIG. 6 shows enzymatic synthesis of thioglycosides 8 and 10. DNP=dinitrophenyl, pNP=para-nitrophenyl.

FIG. 7 shows results of kinetic analysis on Abg E171A with 2,4-DNP β-D-glucopyranoside (125 mM) as donor and with pNp β-D-4-deoxy-4-thio-glucopyranoside as acceptor.

### DETAILED DESCRIPTION OF THE INVENTION

The present invention provides a novel method for the synthesis of S-glycosidic linkages in oligosaccharides by use of glycosidases that lack the catalytic acid/base amino acid residue as a result of a mutation. Glycosidase enzymes can be classified as being either “retainers” because they retain the stereochemistry of the bond being broken during hydrolysis, or “inverters” because they invert the stereochemistry of the bond being broken during hydrolysis. The mutant enzymes of the present invention, which may be referred to herein as thioglycoligases, can be formed by mutation of retaining or inverting, alpha or beta glycosidases.

Normal stereochemistry retaining glycosidase enzymes have two carboxylic acid groups in the active site of the enzyme as shown generally in FIG. 1. One of these groups functions as an acid/base catalyst (labeled as group 1 in FIG. 1) and the other as a nucleophile (group 2 in FIG. 1). The nucleophile group 2 forms a glycosyl-enzyme intermediate which is then cleaved by water or some other nucleophile with the help of the acid/base catalyst group 1 to result in a hydrolyzed glycoside in which the stereochemistry has been maintained.

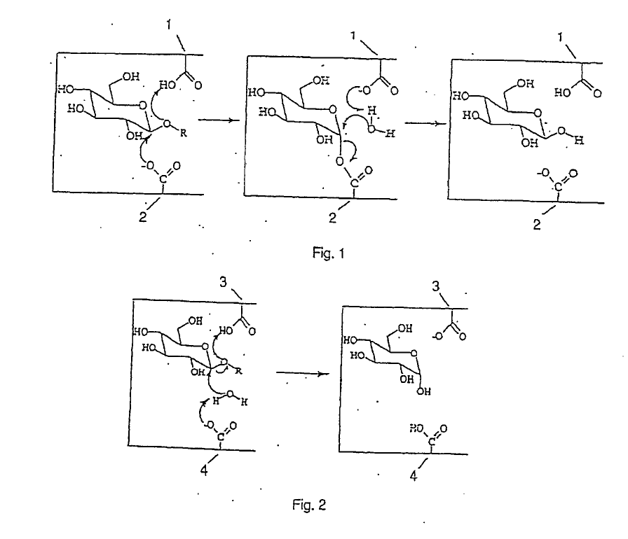

Normal stereochemistry inverting enzymes also have two carboxylic acid groups in the active site of the enzyme as shown generally in FIG. 2. In inverting enzymes, however, one of these groups functions as an acid catalyst (labeled as group 3 in FIG. 2) and the other as a base catalyst (group 4 in FIG. 2). The acid catalyst group 3 protonates the acetal oxygen of the glycosyl donor molecule, making it a good leaving group, at the same time that the base catalyst group 4 deprotonates an acceptor molecule (water or HOR) allowing it to replace the leaving group with inversion of stereochemistry.

The present invention provides mutant forms of both retaining and inverting enzymes in which one of the two carboxylic acid amino acids in the active site has been replaced with a different amino acid. Such mutations provide enzymes which are effective to catalyze the formation of thioglycosides.

Enzymes to which the methodology of the present invention may be employed include, for example, β-glucosidases, β-galactosidases, β-mannosidases, β-N-acetyl glucosaminidases, β-N-acetyl galactosaminidases, β-xylosidases, β-fucosidases, cellulases, xylanases, galactanases, mannanases, hemicellulases, amylases, glucoamylases, α-glucosidases, α-galactosidases, α-mannosidases, α-N-acetyl glucosaminidases, α-N-acetyl galactosaminidases, α-xylosidases, α-fucosidases, neuraminidases/sialidases such as those from: Agrobacterium sp., Bacillus sp., Caldocellum sp., Clostridium sp., Escherichia coli, Kluveromyces sp., Klebsiella sp., Lactobacillus sp., Aspergillus sp., Staphylococcus sp., Lactobacillus sp., Butyrovibrio sp., Ruminococcus sp., Sulfolobus sp., Scihizophyllum sp., Trichoderma sp., Cellulomonas sp., Erwinia sp., Humicola sp., Pseudomonas sp., Thermoascus sp., Phaseolus sp., Persea sp., Fibrobacter sp., Phanaerochaete sp., Microbispora sp., Saccharomyces sp., Hordeum vulgare, Glycine max, Saccharomycopsis sp., Rhizopus sp., Nicotiana, Phaseolus sp., rat, mouse, rabbit, cow, pig, and human sources. Preferred enzymes in accordance with the invention are mutant forms of retaining glycosidase enzymes.

In the enzymes of the present invention, one of the two amino acid residues with the active carboxylic acid side chains is changed to a different amino acid which does not act as an acid/base catalyst (in the case of a retaining enzyme) or as an acid catalyst (in the case of an inverting enzyme). Thus, in general, the substitution will involve replacing the glutamic acid or aspartic acid residue of the wild-type enzyme with alanine, glycine, valine, leucine, isoleucine, serine, threonine, cysteine, methionine, asparagine, glutamine, histidine, proline, phenylalanine, or tyrosine. Preferably, the substituted amino acid will have a side chain of approximately equal or smaller size to the side chain of the wild-type amino acid residue to avoid significant changes to the size and shape of the active site.

Formation of a mutant enzyme with the mutation at the designated amino acid in the active site is suitably performed using site directed mutagenesis to arrive at the desired result. In general, this involves the construction of a plasmid containing the coding sequence for the wild-type gene, and isolation of single stranded DNA. Copies are then made of the isolated plasmid DNA using a template dependant DNA polymerase and a primer which overlaps the site of the desired mutation and which differs from the wild-type sequence in the manner necessary to yield the desired mutation. The mutated plasmid is then transformed into a host organism, e.g., E. coli. Transformants are initially selected using a marker contained within the plasmid, and then further selected by sequencing of the expressed glycosidase enzyme to confirm the nature of the mutation.

Mutant enzymes according to the invention may be purified from the growth medium of the host organism by column chromatography, for example on DEAE-cellulose if desired.

Glycosidases of use in the invention are based upon wild-type enzymes which can be categorized as either alpha or beta glycosidases based upon the anomeric configuration of the natural substrate. When the enzyme modified to form the thioglycoligase used is a retaining enzyme, the resulting thioglycoligase should be used with a donor and acceptor having the same anomeric configuration as each other (for example alpha acceptor with alpha donor) and the same anomeric configuration as the natural substrate. When the enzyme modified to form the thioglycoligase used is a retaining enzyme, the resulting thioglycoligase should be used with an acceptor having the same configuration as the natural substrate and with a donor of opposite configuration.

In accordance with the method of the invention, thioglycosides of the general structure A-S-B, where A and B are both sugar moieties and are formed by the enzymatic coupling of A-X and HS-B using a mutant glycosidase enzyme, wherein X is an appropriate leaving group. In addition to consideration of stereochemistry based on the enzyme being employed, the donors and acceptors are selected based on the desired product, i.e., a glucoside where a glucoside is a desired component, a mannoside where a mannose is a desired component, etc. The specific donor and acceptor are also suitably chosen in light of the double displacement mechanism of retaining glycosidases first proposed by Koshland^{[21]}.

Specific examples of suitable donors include, without limitation, 2,4-dinitrophenyl β-D-glucopyranoside (DNP-Glc); 2,5-dinitrophenyl β-D-mannopyranoside (DNP-Man); DNP β-cellobioside, pNP 4′-deoxy-4′-thio-β-cellobioside and β-D-glucosyl azide. Specific examples of suitable acceptors include, without limitation para-nitrophenyl 4-deoxy-4-thio-β-D-glucopyranoside, para-nitrophenyl 4-deoxy-4thio-β-D-galactopyranoside; methylumbelliferyl 4-deoxy-4-thio-β-D-glucopyranoside, 4′-deoxy-4′-thio-cellobiose, pNP 4′-deoxy-4′-thio-β-cellobioside, and pNP β-D4-deoxy-4-thio-glucopyranoside.

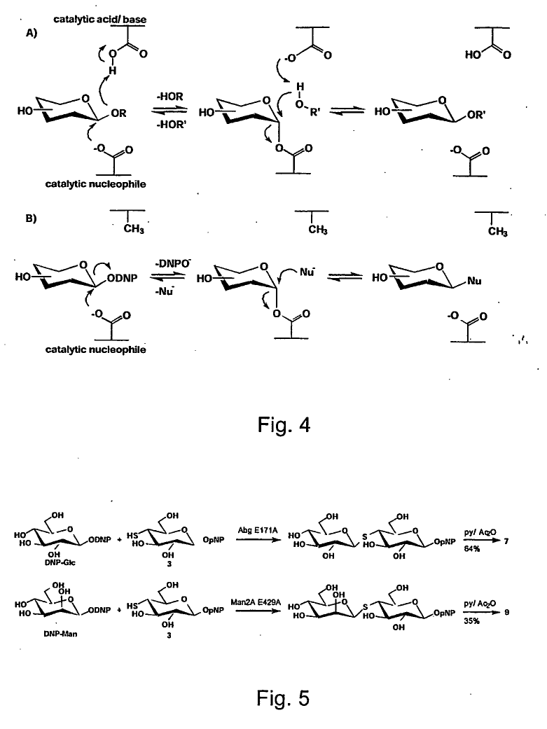

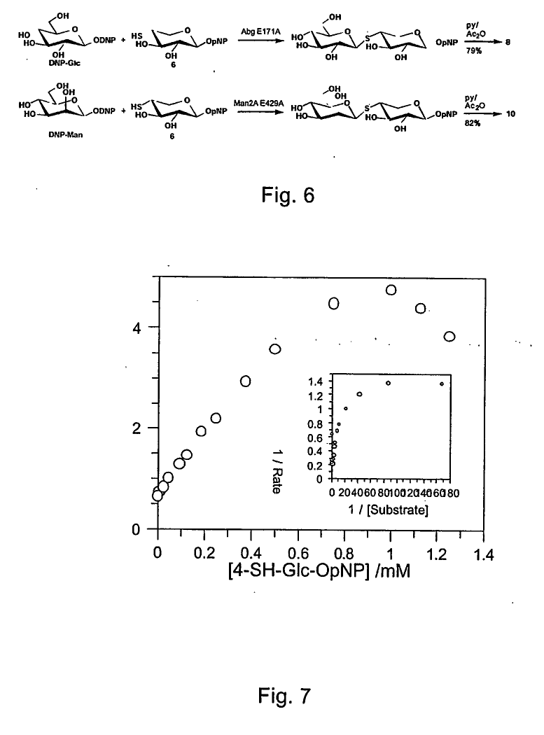

(Scheme 1, FIG. 4): In the glycosylation step the concerted action of the catalytic acid/base (protonated, acting as an acid) and the catalytic nucleophile (deprotonated) leads to the departure of the aglycon group and to the formation of the covalent glycosyl-enzyme intermediate (A). Use of a mutant glycosidase in which the catalytic acid/base residue has been replaced by a non catalytically active residue necessitates glycosyl donors with good leaving groups that do not need acid catalysis, e.g. dinitrophenyl groups, to allow formation of the glycosyl-enzyme intermediate (B). However, turnover of that intermediate via transglycosylation to an oxygen acceptor is impractically slow, since general base catalysis is required to speed this step (A). The acid/base mutant therefore requires strong nucleophiles as acceptors that do not need general base catalysis (B). As a practical consequence, the method of the present invention makes use of a thiosugar acceptor with a free thiol, for example, a para-nitrophenyl 4-deoxy-4-thio glycoside or 3′-deoxy-3′-thio lactose. The thiosugar may be a monosaccharide, an oligosaccharide, or a polysaccharide, and may itself contain thioglycoside linkages as a result of repeated cycles of the method of the invention. The donor species is one having a good leaving group. Typically, such a “good leaving group” will have a leaving group ability from an acetal center equal to or greater than that of p-nitrophenol.

Earlier experiments revealed that small anionic molecules, such as N₃⁻, AcO⁻, HCO₂^{-[22]} and F⁻,^{[23]} rescue the reaction by enhancing the rate of the deglycosylation step. In the present study we exploit this concept to allow the synthesis of thiooligosaccharides by use of deoxythio sugars as nucleophilic acceptors that do not need base catalysis in the deglycosylation step. In summary, our approach requires activated donor glycosides, such as dinitrophenyl glycosides, and chemically synthesized deoxythio sugars as acceptors in conjunction with mutant glycosidases modified at the acid/base position.

We have probed our strategy using the alanine acid/base mutants of two retaining β-glycosidases, the β-glucosidase from Agrobacterium sp. Abg E171A and the β-mannosidase from Cellulomonas fimi Man2A E429A. The mutant Abg E171A was generated by site-directed mutagenesis by a ‘megaprimer’ PCR method using three oligonucleotide primers, one containing the mutation^{[19]} The purified product of the first PCR reaction served as a megaprimer in the second PCR reaction. The purified gene was subcloned into an expression vector, and after expression the protein was purified in a single step by Ni²⁺-chelation chromatography. The mutant Man2A E429A was the same protein sample described by Zechel et al.^{[23]}.

As donors we chose the readily available dinitrophenyl glycosides 2,4-dinitrophenyl β-D-glucopyranoside (DNP-Glc) for studies with the mutant glucosidase and 2,5-dinitrophenyl β-D-mannopyranoside (DNP-Man) for experiments involving the mutant mannosidase.^{[23]} The low pKₐ values of 3.96 for 2,4-dinitrophenol and 5.15 for 2,5-dinitrophenol render acid catalysis for the glycosylation step unnecessary. As an acceptor we initially chose para-nitrophenyl 4-deoxy-4-thio-β-D-glucopyranoside (3) since the parent sugar para-nitrophenyl β-D-glucopyranoside (PNP-Glc) acts as an excellent acceptor for both wild type and nucleophile mutant forms of Abg^{[18]} and Man2^{A}^{[20]} undergoing transfer preferentially to the 4-hydroxyl. The chemical synthesis of the deoxythio sugar 3 was readily achieved via regioselective protection of the galacto sugar, activation of the unprotected axial alcohol by formation of the triflate, nucleophilic substitution with thioacetate with inversion of configuration and finally Zemplen deprotection while excluding oxygen (in the presence of DTT). Anaerobic conditions, even during the enzymatic reactions, are compulsory to prevent oxidation of the thiols to disulfides.

Upon incubation of 3 (20 mM) with the appropriate donor (45 mM) and mutant enzyme (˜1 mg/ml) in phosphate buffered solutions at pH 6.8 TLC analysis revealed that a new product with the expected mobility of a disaccharide was formed quite rapidly in each case. These products were stable towards hydrolysis by both the wild type and mutant enzymes. By contrast, incubation of the wild type enzymes with the appropriate DNP sugar donors and thiosugar acceptor 3 resulted in no significant formation of disaccharide. A novel enzymatic coupling reaction was therefore occurring involving specific thiolinkage formation. This was confirmed by isolation of the products of the enzymatic reactions via silica gel chromatography after acetylation. ¹H and ¹³C NMR analysis as well as mass spectrometry revealed that the products formed were indeed sulfur linked disaccharides (Scheme 2, FIG. 5).

These extremely encouraging results led us to question whether the regiochemical outcome of the transglycosylation reaction could be controlled by virtue of the location of the thiol within the acceptor sugar. We therefore synthesized and tested para-nitrophenyl 4-deoxy-4-thio-β-D-xylopyranoside (6) as an acceptor for the two enzymes. Previous studies with both the wild type Abg and its nucleophile mutant had revealed that para-nitrophenyl β-D-xylopyranoside (PNP-Xyl) is an excellent acceptor substrate, but that transfer, surprisingly, occurred exclusively to the 3-position.^{[18]} Use of 6 as an acceptor would reveal whether the greater nucleophilicity of the thiol controls the reaction outcome. Indeed, incubation of 6 with Abg E171A and DNP-Glc resulted in an excellent yield (79%) of the sulfur-linked disaccharide, as revealed by ¹H and ¹³C NMR analysis as well as ESI-MS. Similarly, incubation of 6 with Man2A E429A and DNP-Man produced, in 82% yield, the β(1-4) linked thiodisaccharide product (Scheme 3).

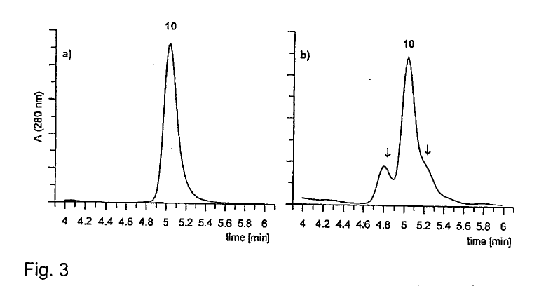

Interestingly, control reactions in which the parent sugars para-nitrophenyl β-D-xylopyranoside (pNP-Xyl) and para-nitrophenyl β-D-glucopyranoside (pNP-Glc) were used as acceptors for Man2A E429A in the presence of DNP-Man, showed the formation of disaccharide products, but only very slowly (However, no formation of disaccharides was observed when using pNP-Glc or pNP-Xyl with Abg E171A in the presence of DNP-Glc as donor). In order to assess the relative rates of transfer to the oxygen and sulfur nucleophiles in the case of Man 2A E429A we established a competition reaction in which equimolar amounts of pNP-Xyl and its 4-thio analogue 6 were incubated together with DNP-Man as donor and Man2A E429A (FIG. 3). When the concentration of donor was limiting (one third of the concentration of acceptors) we saw exclusive formation of the S-linked disaccharide 10 (a). However, upon addition of further donor to a final concentration twice that of the acceptors the O-linked disaccharides (indicated by arrows) were formed (b). Integration of HPLC peaks indicates that transfer to the thiosugar acceptor is at least 100-fold faster than transfer to its oxygen analogue. These results were confirmed by ESI-MS analysis.

The glycosynthases have proven to be powerful tools for the synthesis of O-linked oligosaccharides. In the present invention, the utility of the glycosynthase enzymes is extended and shown to have the further property of being able to produce designed thioglycosides in significant yields. Furthermore, the methodology is general, as demonstrated with two different enzymes from two separate glycosidase families, and will prove to be of considerable value in the specific assembly of thiooligosaccharides for mechanistic and possibly therapeutic use.

A further application of the method of the present invention is the formation of thioglycosidic linkages within proteins in order to generate thioglycosidic analogues of therapeutic glycoproteins, which would be stable to glycosidase-catalysed degradation in vivo. Many therapeutic proteins, such as Epo, TPA, Enbrel, Ceredase, are glycosylated proteins . . . and the glycosylation is important to their function. Typically these proteins have an oligosaccharide on their surface which terminates in a sialyl galactose structure. If all the sialic acids are present the protein has a long circulatory half-life (desirable). However, if any of the sialic acids are missing and the galactose is exposed the protein gets “taken up” by receptors in the liver and cleared from the circulation. Therefore production of glycoproteins that do not lose their sialic acids is desirable because such proteins would have much longer circulatory half-lives. Thioglycosidic modification would provide this benefit. However, at present it is not possible to chemically assemble thioglycosidic bonds right on the protein surface using conventional approaches, as the reagents are too harsh, and no natural enzymes are available to do this job. Mutant thioglycoligase enzymes, however, are perfect for the task.

On a commercial scale, it may be advantageous to immobilize the enzyme to facilitate its removal from a batch of product and subsequent reuse. Such immobilization could be accomplished by use of a fusion protein in which the mutant thioligase is engineered onto another protein with high affinity for an insoluble matrix. For example, a fusion protein with a cellulose binding protein prepared in the manner described by Ong et al., “Enzyme Immobilization Using the Cellulose-Binding Domain of a Cellulomonas fimi Exoglucanase”, Biotechnology 7: 604-607 (1989) could be used in accordance with the invention. Polyhistidine tags may also be used.

The invention will now be further described with reference to the following non-limiting examples:

General Methods:

¹H and ¹³C NMR spectra were recorded on Bruker Avance-300 or Avance-400 Spectrometers. Chemical shifts are reported in δ units (ppm) using residual ¹H and ¹³C signals of the deuterated solvents as reference: δ_{H} (CDCl₃) 7.26, δ_{H} (CD₃OD) 3.31, δ_{C} (CDCl₃) 77.0, δ_{C} (CD₃OD) 49.0. Electrospray mass spectra were recorded on a PE Sciex API 300 LC/MS/MS instrument by direct injection of the compounds in a 1:1 CH₃CN/H₂O solution. Melting points were determined with a Mel-Temp II apparatus and are not corrected. Silica gel 60 (230-400 mesh) from SiliCycle was used for column chromatography. The petroleum ether used for column chromatography had a boiling point range from 35-60° C. Amberlite IR-120PLUS from Aldrich was transformed into the H⁺-form before use. All reagents and solvents were purchased from Aldrich, Fluka, Sigma or Fisher Scientific. Solvents were dried over CaH₂ (CH₂Cl₂, pyridine, toluene, acetonitrile), over Mg (methanol) or over molecular sieves 4 Å (DMF). All reactions were carried out under a dry nitrogen atmosphere.

### EXAMPLE 1

### Chemical Synthesis of Deoxythio Sugar Acceptors

### p-Nitrophenyl 2,3,6-tri-O-benzoyl-β-D-galactopyranoside (1)

Benzoyl chloride (1.50 ml, 1.82 g, 12.9 mmol) was added dropwise to a solution of p-nitrophenyl β-D-galactopyranoside (1.00 g, 3.32 mmol) in DMF (15 ml) and pyridine (15 ml) at −20° C. After stirring for 5 h at −5° C. another 0.30 ml of benzoyl chloride (0.36 g, 2.59 mmol) was added dropwise, and the solution was stirred for 2 h at −5° C. Water (10 ml) was added and the mixture was concentrated by evaporation in vacuo. The residue was dissolved in CH₂Cl₂, and the organic phase was washed sequentially with saturated aqueous NaHCO₃, 1 M HCl and brine, dried over MgSO₄, filtered and concentrated in vacuo. Column chromatography (toluene→4:1 toluene/EtOAc) followed by crystallization from hot toluene yielded 1 (850 mg, 1.39 mmol, 42%); mp 180-181° C.; ¹H-NMR (400 MHz): δ_{H} (CDCl₃): 8.05 (m, 2 H, Ar), 8.0-7.3 (m, 15 H, 3xBz), 7.06 (m, 2 H, Ar), 6.10 (dd, 1 H, J_{2,3} 10.3 Hz, J_{2,1} 7.9 Hz, H-2), 5.48 (dd, 1 H, J_{3,2} 10.3 Hz, J_{3,4} 3.2 Hz, H-3), 5.40 (d, 1 H, J_{1,2} 7.9 Hz, H-1), 4.76 (dd, 1 H, J_{6,6} 11.7 Hz, J_{6,5} 5.1 Hz, H-6), 4.68 (dd, 1 H, J_{6,6} 11.7 Hz, J_{6,5} 7.7 Hz, H-6), 4.47 (m, 1 H, H-4), 4.31 (m, 1 H, H-5), 2.66 (d, 1 H, J_{OH,4} 4.5 Hz, OH); ¹³C-NMR (75 MHz): δ_{C} (CDCl₃): 166.4, 165.8, 165.3, 161.3, 143.0, 133.7, 133.5, 129.9, 129.7, 129.7, 129.3, 129.0, 128.6, 128.6, 128.5, 128.5, 125.6, 116.8, 98.8, 73.9, 73.2, 69.0, 67.1, 63.0; ESI-MS: m/z=636.5 [M+Na]⁺ (expected for C₃₃H₂₇NO₁₁Na⁺: m/z=636.2).

### p-Nitrophenyl 4-S-acetyl-2,3,6-tri-O-benzoyl-4-deoxy-4-thio-β-D-glucopyranoside (2)

Trifluoromethanesulfonic anhydride (0.44 ml, 0.75 g, 2.7 mmol) was added dropwise to a solution of 1 (813 mg, 1.33 mmol) in 20 ml CH₂Cl₂ and 1.2 ml pyridine at 0° C. After 1 h at 0° C., CH₂Cl₂ (50 ml) was added, and the organic layer was washed with saturated aqueous NaHCO₃, 1M HCl and brine, dried over MgSO₄, filtered and concentrated in vacuo to give 990 mg of a yellowish solid (100%). Potassium thioacetate (460 mg, 4.0 mmol) and HMPA (10 ml) were added, and the suspension was stirred at RT for 1 h. A mixture of EtOAc/Et₂O (1:1, 50 ml) was added, and the organic layer was washed twice with water, with brine, dried over MgSO₄, filtered and concentrated in vacuo. Column chromatography (19:1→3:1 PE/EtOAc) and crystallization from hot EtOAc yielded 2 as a white powder (520 mg, 59%); mp 249° C. (degradation); ¹H-NMR (300 MHz): δ_{H} (CDCl₃): 8.06 (m, 2 H, Ar), 8.02-7.31 (m, 15 H, 3xBz), 7.02 (m, 2 H, Ar), 5.84 (dd, 1 H, J_{3,4} 10.8 Hz, J_{3,2} 9.3 Hz, H-3), 5.73 (dd, 1 H, J_{2,3} 9.3 Hz, J_{2,1} 7.6 Hz, H-2), 5.43 (d, 1 H, J_{1,2} 7.6 Hz, H-1), 4.79 (dd, 1 H, J_{6,6} 12.0 Hz, J_{6,5} 2.2 Hz, H-6), 4.54 (dd, 1 H, J_{6,6} 12.0 Hz, J_{6,5} 7.2 Hz, H-6), 4.37 (m, 1 H, H-5), 4.10 (t, 1 H, J_{4,5}=J_{4,3} 10.8 Hz, H-4), 2.28 (s, 3 H, Ac); ¹³C-NMR (75 MHz): δ_{C} (CDCl₃): 192.7, 165.9, 165.6, 165.0, 161.1, 143.1, 138.8, 133.6, 133.6, 129.9, 129.8, 129.7, 129.4, 128.8, 128.6, 128.5, 128.4, 125.6, 116.8, 98.3, 73.7, 72.5, 71.2, 63.7, 44.3, 30.8; ESI-MS: m/z=694.0 [M+Na]⁺ (expected for C₃₅H₂₉NO₁₁SNa⁺: m/z=694.1).

### p-Nitrophenyl 4-deoxy-4-thio-β-D-glucopyranoside (3)

A solution of 2 (230 mg, 0.34 mmol) in 10 ml MeOH containing catalytic amounts of MeONa was stirred for 3 h at RT. The mixture was neutralized with Amberlite IR-120PLUS (H⁺-form). After filtration, DTT (280 mg, 1.8 mmol) in 2 ml of degassed water was added, and N₂ was bubbled through the solution for 5 min. After stirring under N₂ overnight the mixture was concentrated in vacuo. Column chromatography (3:2 toluene/EtOAc→EtOAc) afforded 3 as a white powder (80 mg, 0.25 mmol, 74%); ¹H-NMR (400 MHz): δ_{H} (d₄-MeOH): 8.22 (m, 2 H, Ar), 7.23 (m, 2 H, Ar), 5.10 (d, 1 H, J_{1,2} 7.6 Hz, H-1), 3.94 (dd, 1 H, J_{6,5} 12.3 Hz, J_{6,6} 1.9 Hz, H-6), 3.84 (dd, 1 H, J_{6,6} 12.3 Hz, J_{6,5} 4.8 Hz, H-6), 3.62 (m, 1 H, H-5), 3.49 (dd, 1 H, J_{2,3} 9 Hz, J_{2,1} 7.6, H-2), 3.41 (dd, 1 H, J_{3,4} 10.2 Hz, J_{3,2} 9.0 Hz, H-3), 2.84 (t, 1 H, J_{4,5}=J_{4,3} 10.2 Hz, H-4); ¹³C-NMR (75 MHz) δ_{C} (d₄-MeOH): 163.9, 143.9, 126.6, 117.7, 101.5, 79.8, 78.8, 75.7, 62.9, 43.0; ESI-MS: m/z=340.0 [M+Na]⁺ (expected for C₁₂H₁₅NO₇SNa⁺: m/z=340.1).

### p-Nitrophenyl 2,3-di-O-benzoyl-α-L-arabinopyranoside (4)

Benzoyl chloride (1.0 ml, 1.25 g, 8.7 mmol) was added dropwise to a solution of p-nitrophenyl α-L-arabinopyranoside (1.00 g, 3.8 mmol) in DMF (30 ml) and pyridine (10 ml) at −20° C. The mixture was allowed to warm to RT overnight while stirring and worked up as compound 1. Column chromatography (5:1→2:1 PE/EtOAc) and crystallization from EtOAc/heptane yielded 4 as a white powder (520 mg, 1.11 mmol, 29%); mp 150-151° C.; ¹H-NMR (400 MHz): δ_{H} (CDCl₃): 8.18 (m, 2 H, Ar), 8.13-7.40 (m, 10 H, 2xBz), 7.08 (m, 2 H, Ar), 5.80 (dd, 1 H, J_{2,3} 6.4 Hz, J_{2,1} 4.2 Hz, H-2), 5.55 (dd, 1 H, J_{3,2} 6.4 Hz, J_{3,4} 3.3 Hz, H-3), 5.52 (d, 1 H, J_{1,2} 4.2 Hz, H-1), 4.46 (m, 1 H, H-4′), 4.17 (dd, 1 H, J_{5,5} 11.9 Hz, J_{5,4} 7.0 Hz, H-5), 3.90 (dd, 1 H, J_{5,5} 11.9 Hz, J_{5,4} 3.4 Hz, H-5), 2.40 (d, 1 H, J_{OH,4} 5.3 Hz, OH); ¹³C-NMR (100 MHz): δ_{C} (CDCl₃): 165.9, 165.0, 161.2, 142.9, 133.8, 133.7, 129.9, 129.8, 128.8, 128.6, 128.6, 125.8, 116.5, 96.8, 71.4, 69.0, 65.1, 62.9; ESI-MS: m/z=502.0 [M+Na]⁺ (expected for C₂₅H₂₁NO₉Na⁺: m/z=502.1).

### p-Nitrophenyl 4-S-acetyl-2,3-di-O-benzoyl-4-deoxy-4-thio-β-D-xylopyranoside (5)

The partially protected glycoside 4 (340 mg, 0.71 mmol) was treated as described for the preparation of 2. Column chromatography (6:1→2:1 PE/EtOAc) gave xyloside 5 as a white foam (215 mg, 0.4 mmol, 56%); ¹H-NMR (400 MHz): δ_{H} (CDCl₃): 8.19 (m, 2 H, Ar), 8.1-7.35 (m, 10 H, 2xBz), 7.09 (m, 2 H, Ar), 5.60 (m, 1 H, H-3), 5.57 (m, 1 H, H-2), 5.50 (d, 1 H, J_{1,2} 4.9 Hz, H-1), 4.42 (dd, 1 H, J_{5,5} 12.2 Hz, J_{5,4} 4.2 Hz, H-5), 4.07 (m, 1 H, H-4), 3.75 (dd, 1 H, J_{5,5} 12.2 Hz, J_{5,4} 7.5 Hz, H-5), 2.35 (s, 3 H, Ac); ¹³C-NMR (100 MHz): δ_{C} (CDCl₃): 193.4, 165.2, 165.0, 161.1, 143.0, 133.6, 129.9, 128.9, 128.8, 128.5, 125.8, 116.6, 97.7, 70.2, 69.8, 63.4, 41.3, 30.7; ESI-MS: m/z=560.0 [M+Na]⁺ (expected for C₂₇H₂₃NO₉SNa⁺: m/z=560.1).

### p-Nitrophenyl 4-deoxy-4-thio-β-D-xylopyranoside (6)

The protected 4-deoxy-4-thioxyloside 5 (190 mg, 0.35 mmol) was deprotected as described for compound 3. Column chromatography (1:1→1:3 PE/EtOAc) gave 6 as a white powder (70 mg, 0.24 mmol, 69%); ¹H-NMR (400 MHz): δ_{H} (d₄-MeOH): 8.20 (m, 2 H, Ar), 7.18 (m, 2 H, Ar), 5.03 (d, 1 H, J_{1,2} 7.6 Hz, H-1), 3.98 (dd, 1 H, J_{5,5} 11.8 Hz, J^{5,4} 5.0 Hz, H-5), 3.52 (t, 1 H, J_{5,5}=J_{5,4} 11.8 Hz, H-5), 3.44 (dd, 1 H, J_{2,3} 8.9 Hz, J_{2,1} 7.6 Hz, H-2), 3.32 (m, 1 H, H-3), 2.84 (m, 1 H, H-4); ¹³C-NMR (100 MHz): δ_{C} (d₄-MeOH): 163.7, 143.9, 126.6, 117.6, 102.3, 78.6, 75.9, 69.2, 42.0; ESI-MS: m/z=310.0 [M+Na]⁺ (expected for C₁₁H₁₃NO₆SNa⁺: m/z=310.0).

### Enzymatic Synthesis of Thiooligosaccharides

The deoxythio sugars 3 or 6 (20 mM), the DNP-donors 2,4-dinitrophenyl β-D-glucopyranoside (DNP-Glc) or 2,5-dinitrophenyl β-D-mannopyranoside (DNP-Man) (30 mM) and the mutant enzymes Abg E171A or Man2A E429A (˜1 mg ml⁻¹) were incubated for ˜3 h at RT in phosphate buffer (80 mM). DNP-Glc or DNP-Man was added to a total concentration of 45 mM, and the solution was incubated at RT for ˜1 h. After lyophilization standard per-O-acetylation with pyridine/Ac₂O and subsequent workup was performed. The final purification by column chromatography (9:1→1:1 toluene/EtOAc) yielded products 7-10.

### p-Nitrophenyl (2,3,4,6-tetra-O-acetyl-β-D-glucopyranosyl)-(1→4)-S-2,3,6-tri-O-acetyl-4-deoxy-4-thio-β-D-glucopyranoside (7)

25 mg (64%); mp 161.5-162° C. (hot toluene); ¹H-NMR (400 MHz): δ_{H} (CDCl₃): 8.19 (m, 2 H, Ar), 7.09 (m, 2 H, Ar), 5.29-5.16 (m, 4 H, H-1, H-2, H-3), 5.06 (t, 1 H, J_{4′,3′}=J_{4′,5′}9.8 Hz, H-4′), 4.94 (t, 1 H, J_{2′,1′}=J_{2′,3′}9.6 Hz, H-2′), 4.77 (d, 1 H, J_{1′,2′}10 Hz, H-1′), 4.64 (dd, 1 H, J_{6,6} 12.1 Hz, J_{6,5} 1.7 Hz, H-6), 4.38 (dd, 1 H, J_{6,6} 12.1 Hz, J_{6,5} 5.5 Hz, H-6), 4.31 (dd, 1 H, J_{6′,6′}12.4 Hz, J_{6′,5′}2.2 Hz, H-6′), 4.13 (dd, 1 H, J_{6′,6′}12.4 Hz, J_{6′,5′}4.8 Hz, H-6′), 4.06 (m, 1 H, H-5), 3.75 (m, 1 H, H-5′), 3.05 (t, 1 H, J_{4,5}=J_{4,3} 10.6 Hz, H-4), 2.10, 2.09, 2.07, 2.06, 2.04, 2.02, 2.00 (7xs, 21 H, 7xAc); ¹³C-NMR (100 MHz): δ_{C} (CDCl₃): 170.4, 170.1, 170.0, 170.0, 169.3, 169.3, 169.2, 161.3, 143.2, 125.7, 116.7, 98.0, 81.7, 75.9, 74.6, 73.6, 72.4, 70.3, 69.9, 68.1, 63.3, 61.9, 45.8, 20.7-20.4 (7x); ESI-MS: m/z=796.0 [M+Na]⁺ (expected for C₃₂H₃₉NO₁₉SNa⁺: m/z=796.2).

### p-Nitrophenyl (2,3,4,6-tetra-O-acetyl-β-D-glucopyranosyl)-(1→4)-S-2,3-di-O-acetyl-4-deoxy-4-thio-βD-xylopyranoside (8)

29 mg (79%); mp 122-123° C. (EtOAc/heptane); ¹H-NMR (400 MHz): δ_{H} (CDCl₃): 8.20 (m, 2 H, Ar), 7.07 (m, 1 H, Ar), 5.21-5.11 (m, 4 H, H-1, H-2, H-3, H-3′), 5.07 (t, 1 H, J_{4′,3′}=J_{4′,5′}9.9 Hz, H-4′), 5.00 (dd, 1 H, J_{2′,1′}10 Hz, J_{2′,3′}9.3 Hz, H-2′), 4.63 (d, 1 H, J_{1′,2′}10 Hz, H-1′), 4.29 (dd, 1 H, J_{5′,5′}12.4 Hz, J_{5,4} 4.5 Hz, H-5), 4.19 (d, 2 H, J_{6′,5′}3.5 Hz, 2xH-6′), 3.74 (dt, 1 H, J_{5′,4′}9.9 Hz and J_{5′,6′}3.5 Hz, H-5′), 3.65 (dd, 1 H, J_{5,5} 12.4 Hz, J_{5,4} 9.6 Hz, H-5), 3.23 (m, 1 H, H-4), 2.10, 2.06, 2.05, 2.03, 2.02, 2.00 (6xs, 18 H, 6xAc); ¹³C-NMR (100 MHz): δ_{C} (CDCl₃): 170.4, 170.0, 169.9, 169.2, 169.2, 169.0, 161.1, 143.0, 125.7, 116.5, 98.1, 81.9, 76.0 (x2), 73.6, 71.3, 69.9, 67.9, 65.6, 61.8, 42.7, 20.5 (6x); ESI-MS: m/z=724.5 [M+Na]⁺ (expected for C₂₉H₃₅NO₁₇SNa⁺: m/z=724.2).

### p-Nitrophenyl (2,3,4,6tetra-O-acetyl-β-D-mannopyranosyl)-(1→1)-S-2,3,6-tri-O-acetyl-4-deoxy-4-thio-β-D-glucopyranoside (9)

5 mg (35%); ¹H-NMR (400 MHz): δ_{H} (CDCl₃): 8.20 (m, 2 H, Ar), 7.09 (m, 2 H, Ar), 5.36 (dd, 1 H, J_{2′,3′}3.5 Hz, J_{2′,1′}1 Hz, H-2′), 5.32-5.17 (m, 4 H, H-1, H-2, H-3, H-4′), 5.09 (dd, 1 H, J_{3′,4′}10.1 Hz, J_{3′,2′}3.5 Hz, H-3′), 4.98 (d, 1 H, J_{1′,2′}1 Hz, H-1′), 4.61 (dd, 1 H, J_{6,6} 12.2 Hz, J_{6,5} 2.1 Hz, H-6), 4.46 (dd, 1 H, J_{6,6} 12.2 Hz, J_{6,5} 5.4 Hz, H-6), 4.33 (dd, 1 H, J_{6′,6′}12.4 Hz, J_{6′,5′}2.4 Hz, H-6′), 4.15 (dd, 1 H, J_{6′,6′}12.4 Hz, J_{6′,5′}5.5 Hz, H-6), 4.08 (m, 1 H, H-5), 3.75 (m, 1 H, H-5), 3.07 (t, 1 H, J_{4,5}=J_{4,3} 10.7 Hz, H-4), 2.20 2.12, 2.10, 2.07, 2.06, 2.06, 1.98 (7xs, 21 H, 7xAc); ¹³C-NMR (75 MHz): δ_{C} (CDCl₃): 170.5, 170.3, 170.3, 170.0, 169.8, 169.6, 169.2, 161.2, 143.2, 125.7, 116.6, 98.1, 79.3, 74.3, 72.1, 71.5, 69.9, 69.7, 65.7, 45.1, 20.6 (7x); ESI-MS: m/z=796.0 [M+Na]⁺ (expected for C₃₂H₃₉NO₁₉SNa⁺: m/z=796.2).

### p-Nitrophenyl (2,3,4,6-tetra-O-acetyl-β-D-mannopyranosyl)-(1→4)-S-2,3-di-O-acetyl-4-deoxy-4-thio-β-D-xylopyranoside (10)

25 mg (82%); ¹H-NMR (400 MHz): δ_{H} (CDCl₃): 8.20 (m, 2 H, Ar), 7.07 (m, 2 H, Ar), 5.45 (d, 1 H, J_{2′,3′}3.5 Hz, H-2′), 5.26-5.12 (m, 4 H, H-1, H-2, H-3, H-4′), 5.07 (dd, 1 H, J_{3′,4′}10.1 Hz, J_{3′,2′}3.5 Hz, H-3′), 4.87 (s, 1 H, H-1′), 4.28-4.15 (m, 3 H, H-5, H-6′, H-6′), 3.75 (m, 1 H, H-5′), 3.66 (dd, 1 H, J_{5,5} 12.4 Hz, J_{5,4} 10.0 Hz, H-5), 3.28 (m, 1 H, H-4), 2.20, 2.10, 2.08, 2.06, 2.04, 2.03, 1.97 (6xs, 18 H, 6xAc); ¹³C-NMR (75 MHz): δ_{C} (CDCl₃): 170.4, 170.2, 170.0, 169.9, 169.6, 169.3, 161.1, 143.1, 125.8, 116.5, 98.3, 79.8, 76.8, 71.7, 71.5, 70.9, 69.8, 65.5 (2x), 62.6, 43.0, 20.5 (6x); ESI-MS: m/z=724.5 (M+Na]⁺ (expected for C₂₉H₃₅NO₁₇SNa⁺: m/z=724.2).

### EXAMPLE 2

### Competition Study for Evaluation of Relative Rates

pNP-Xyl (5.5 mM), 6 (5.5 mM), DNP-Man (3.75 mM) and Man2A E429A (˜1 mg ml⁻¹) were incubated in phosphate buffer (50 mM, pH 6.8) for 3 h at RT. After removal of an aliquot DNP-Man was added to a final concentration of 22 mM. After 5 h at RT another aliquot was taken. The aliquots were diluted 1:3 with acetonitrile and centrifuged before applying to the HPLC. HPLC analysis was performed using a Waters 600E multisolvent delivery system with acetonitrile (A)/water (B) as mobile phase (linear gradient: 80% A→60% A in 15 min. flow: 1 ml min⁻¹), a Waters 2486 Dual λ Absorbance Detector (detection at 280 nm), a TOSO HAAS Amide 80 column (4.6×250 mm) and Millenium 3.20 software.

### EXAMPLE 3

Kinetic analysis was performed with 2,4-DNP β-D-glucopyranoside (125 mM) as donor and with pNP 4-deoxy-4-thio-β-D-glucopyranoside as acceptor at 30° C. in 50 mM NaPi, 145 mM NaCl pH 7.1 (FIG. 7). It has been shown previously, that the K_{M}-value of the donor is extremely low (<1 μM), and that no donor substrate inhibition is seen. Enzymatic background hydrolysis of the donor together with acceptor substrate inhibition make it difficult to exactly determine K′ₘ and k′_{cat}, but it can be calculated from the highest observed rate of DNP release, that k′_{cat} is higher than 50 min⁻¹.

### EXAMPLE 4

Saturation mutagenesis at the acid/base position (E171) of Abg revealed new functional mutations with functionality as thioligases as follows: Abg E171G, Abg E171Q, E171S, E171T, E171M, E171F, E171L, E171I, E171N.

### EXAMPLE 5

An endo acting retaining β-glycosidase from Cellulomonas fimi (Cex) was successfully transformed into a functional thioglycoligase by mutation of the catalytic acid/base residue E127. This is especially interesting, as the conversion of Cex into a glycosynthase showed no success. Functional donors for Cex E127A were DNP β-cellobioside and pNP 4′-deoxy-4′-thio-β-cellobioside, the latter resulting in self condensation to form the thiolinked tetrasaccharide. Functional acceptors were 4′-deoxy-4′-thio-cellobiose, pNP 4′-deoxy-4′-thio-b-cellobioside and pNP β-D-4-deoxy-4-thio-glucopyranoside.

### EXAMPLE 6

The endo mannanase Man26A from Cellvibrio japonicus has been transformed into the acid/base mutant E212A. TLC and ESI-MS experiments indicate, that Man26A E212A is a functional thioglycoligase catalyzing the transglycosylation reaction of DNP mannobioside with pNP 4′-deoxy-4′-thio-b-cellobioside:

### EXAMPLE 7

Cloning of Abg acid/base mutant: The E171A mutation was introduced using the megaprimer method and pET29AbgHis6 (Mayer et al., FEBS Letters, 466, 40-44) as template. The primers used were p29fwd:
5′ GGG GAC AAG TTT GTA CAA AAA AGC AGG CTA AGA AGG AGA TAT ACA TAT G (Seq. ID. No. 1) and AbgE171A:

5′ CGC GCA CCA AGG CGC GTT GAA CGT TGC AAC CGC ATC (Seq. ID No. 2) in the first reaction. For the second reaction the product of the first PCR was use as megaprimer in combination with p29rev:

| | |
|---|---|
| 5′ GGG GAG GAG TTT GTA CAA GAA AGC | (Seq. ID. No 3) |
| TGG GTC TGA GTG GTG GTG GTG GTG. | |

The resulting product was inserted into the pDONR201 vector and subsequently transferred to pDEST14 using the Gateway cloning system (Gateway Cloning Technology Manual Version 1. GibcoBRL Life Technologies). Protein expression and purification was performed as described in Mayer et al.(Mayer et al., FEBS Letters, 466, 40-44).

### REFERENCES

The following references, all of which are incorporated herein by reference, are cited herein:
1. Witczak, Z. J., Boryczewski, D., Bioorg. Med. Chem. Lett. 1998, 8, 3265-3268
2. Blanc-Meusser, M., Vigne, L., Driguez, H., Lehmann, J., Steck, J., Urbahns, K., Carbohydr. Res. 1992, 224, 59-71
3. Schou, C., Rasmussen, G., Schuelein, M., Henrissat, B., Driguez, H., J. Carbohydr. Chem 1993, 12, 743-752
4. Sulzenbacher, G., Driguez, H., Henrissat, B., Schuelein, M., Davies, G. J., Biochemistry 1996, 35, 15280-15287
5. Czjzek, M., Cicek, M., Zamboni, V., Burmeister, W. P., Bevan, D. R., Henrissat, B., Esen, A., Biochem J. 2001, 354, 37-46
6. Hrmova, M., Varghese, J. N., De Gori, R, Smith, B. J., Driguez, H., Fincher, G. B., Structure 2001, 9, 1005-1016
7. Varrot, A., Schulein, M., Fruchard, S., Driguez, H., Davies, G. J., Acta Cryst. 2001, D 57, 1739-1742
8. Watson, K. A., McCleverty, C., Geremia, S., Cottaz, S., Driguez, H., Johnson, L. N., EMBO J. 1999, 18, 4619-4632
9. Witczak, Z. J., Curr. Med. Chem 1999, 6, 165-178
10. Andrews, J. S., Pinto, B. M., Carbohydr. Res. 1995, 270, 51-62
11. Wang, L. X., Sakairi, N., Kuzuhara, H., J. Chem Soc. Perkin Trans. 1 1990, 1677-1682
12. Blanc-Muesser, M., Defaye, J., Driguez, H., Carbohydr. Res. 1978, 67, 305-328
13. Fort, S., Varrot, A., Schulein, M., Cottaz, S., Driguez, H., Davies, G. J., Chembiochem 2001, 2, 319-325
14. Witczak, Z. J., Sun, J. M., Mielguj, R., Bioorg. Med. Chem. Lett. 1995, 5, 2169-2174
15. review: Fahey, J. W., Zalcmann, A. T., Talalay, P., Phytochemistry 2001, 56, 5-51
16. GrootWassink, J. W. D., Reed, D. W., Kolenovsky, A. D., Plant. Physiol. 1994, 105, 425-433
17. Marillia, E. F., MacPherson, J. M., Tsang, E. W. T., Van Audenhove, K., Keller, W. A., GrootWassink, J. W. D., Physiol. Plantarum 2001, 113, 176-184
18. Mackenzie, L. F., Wang, Q. P., Warren, R. A. J., Withers, S. G., J. Am. Chem. Soc. 1998, 120, 5583-5584
19. Mayer, C., Zechel, D. L., Reid, S. P., Warren, R. A. J., Withers, S. G., FEBS Lett. 2000, 466, 40-44
20. Nashiru, O., Zechel, D. L., Stoll, D., Mohammadzadeh, T., Warren, R. A. J., Withers, S. G., Angew. Chem Int. Edit 2001, 40, 417-420
21. Koshland, D. E., Biol. Rev. 1953, 28, 416-436
22. Wang, Q., Trimbur, D., Graham, R., Warren, R. A. J., Withers, S. G., Biochemistry 1995, 34, 14554-14562
23. Zechel, D. L., Reid, S. P., Nashiru, O., Mayer, C., Stoll, D., Jakeman, D. L., Warren, R. A. J., Withers, S. G., J. Am. Chem. Soc. 2001, 123, 4350-4351

1-19. (canceled)

20. A mutant form of a glycosidase enzyme, said enzyme being selected from among glycosidase enzymes having two catalytically active amino acids with carboxylic acid side chains within the active site of the wild-type enzyme including a catalytically active amino acid acting as an acid, base, or acid/base catalyst, said mutant enzyme being mutated to replace the catalytically active amino acid acting as an acid, base or acid/base catalyst with a different amino acid having a non-carboxylic acid side chain.

21. The enzyme of claim 20, wherein the different amino acid has a side chain that is approximately equal in size to or smaller than the smaller chain of the replaced amino acid.

22. The enzyme of claim 21, wherein the different amino acid is selected from the group consisting of alanine, glycine, valine, leucine, isoleucine, serine, threonine, cysteine, methionine, asparagine, glutamine, histidine, proline, phenylalanine, and tyrosine.

23. The enzyme of claim 20, wherein the mutant enzyme is formed by replacing the amino acid in the active site of an enzyme selected from the group consisting of β-glucosidases, β-galactosidases, β-mannosidases, β-N-acetyl glucosaminidases, β-N-acetyl galactosaminidases, β-xylosidases, β-fucosidases, cellulases, xylanases, galactanases, mannanases, hemicellulases, amylases, glucoamylases, α-glucosidases, α-galactosidases, α-mannosidases, α-N-acetyl glucosaminidases, α-N-acetyl galactosaminidases, α-xylosidases, α-fucosidases, and neuraminidases/sialidases.

24. The enzyme of claim 20, wherein the mutant enzyme is a mutant of Agrobacterium β-glucosidase.

25. The enzyme of claim 24, wherein the mutant enzyme is selected from the group consisting of AbgE171A, E171G, E171Q, E171S, E171T, E171M, E171F, E171L, E171I, and E171N.

26. The enzyme of claim 20, wherein the mutant enzyme is a mutant of an endo-acting retaining β-glycosidase of Cellulomonas fimi.

27. The enzyme of claim 26, wherein the mutant enzyme is Cex E127A.

28. The enzyme of claim 20, wherein the mutant enzyme is a mutant of an endo-mannanase Man26A of Cellvibrio japonicus.

29. The enzyme of claim 28, wherein the mutant enzyme is Man26A E212A.

30. A method for synthesizing a thioglycoside having the structure A-S-B, wherein S is sulfur and A and B are each sugar moieties, comprising the steps of:
(a) combining a donor molecule A-X, where X is a leaving group, and an acceptor molecule HS-B in a reaction mixture; and
(b) enzymatically coupling the donor molecule to the acceptor molecule using a mutant form of a glycosidase enzyme, said enzyme being selected from among glycosidase enzymes having two catalytically active amino acids with carboxylic acid side chains within the active site of the wild-type enzyme including a catalytically active amino acid acting as an acid, base, or acid/base catalyst, said mutant enzyme being mutated to replace the catalytically active amino acid acting as an acid, base or acid/base catalyst with a different amino acid having a non-carboxylic acid side chain.

31. The method of claim 30, wherein the leaving group X is dinitrophenol.

32. The method of claim 30, wherein the donor is selected from the group consisting of 2,4-dinitrophenyl β-D-glucopyranoside (DNP-Glc); 2,5-dinitrophenyl β-D-mannopyranoside (DNP-Man); DNP β-cellobioside, pNP 4′-deoxy-4′-thio-β-cellobioside and β-D-glucosyl azide.

33. The method of claim 30, wherein the acceptor is selected from the group consisting of para-nitrophenyl 4-deoxy-4-thio-β-D-glucopyranoside, para-nitrophenyl 4-deoxy-4-thio-β-D-galactopyranoside; methylumbelliferyl 4-deoxy-4-thio-β-D-glucopyranoside, 4′-deoxy-4′-thio-cellobiose, pNP 4′-deoxy-4′-thio-β-cellobioside, and pNP β-D-4-deoxy-4-thio-glucopyranoside.

34. The method of claim 30, wherein the glycosidase enzyme is a stereochemistry inverting enzyme in which one of the carboxylic acid side chains in the active site functions as an acid catalyst and the other carboxylic acid side chain functions as a base catalyst, and wherein the amino acid having the carboxylic acid side chain which functions as an acid catalyst is replaced in the mutant enzyme.

35. The method of claim 30, wherein the glycosidase enzyme is a stereochemistry retaining enzyme in which one of the carboxylic acid side chains in the active site functions as an acid/base catalyst and the other carboxylic acid side chain functions as a nucleophile, and wherein the amino acid having the carboxylic acid side chain which functions as an acid/base catalyst is replaced in the mutant enzyme.

36. The method of claim 30, wherein the mutant enzyme is a mutant of Agrobacterium β-glucosidase, an endo-acting retaining β-glycosidase of Cellulomonas fimi or an endo-mannanase Man26A of Cellvibrio japonicus.

37. A thioglycoside prepared by the method of claim 30.

38. A fusion protein comprising
(a) a mutant form of a glycosidase enzyme, said enzyme being selected from among glycosidase enzymes having two catalytically active amino acids with carboxylic acid side chains within the active site of the wild-type enzyme including a catalytically active amino acid acting as an acid, base, or acid/base catalyst, said mutant enzyme being mutated to replace the catalytically active amino acid acting as an acid, base or acid/base catalyst with a different amino acid having a non-carboxylic acid side chain, and
(b) a binding element for immobilization of the fusion protein on a solid support.

39. The fusion protein of claim 38, wherein the binding element is the cellulose-binding domain of a Cellulomonas fimi exoglucanase.

40. The fusion protein of claim 39, wherein the mutant enzyme is a mutant of Agrobacterium β-glucosidase, an endo-acting retaining β-glycosidase of Cellulomonas fimi or an endo-mannanase Man26A of Cellvibrio japonicus.

41. The fusion protein of claim 38, wherein the mutant enzyme is a mutant of Agrobacterium β-glucosidase, an endo-acting retaining β-glycosidase of Cellulomonas fimi or an endo-mannanase Man26A of Cellvibrio japonicus.


SEQUENCE LISTING (3 sequences)

SEQ ID NO: 1 - DNA, 49 bp, artificial
  P29 forward megaprimer
ggggacaagtttgtacaaaaaagcaggctaagaaggagatatacatatg

SEQ ID NO: 2 - DNA, 36 bp, Agrobacterium sp.
cgcgcaccaaggcgcgttgaacgttgcaaccgcatc

SEQ ID NO: 3 - DNA, 48 bp, artificial
  p29 reverse megaprimer
ggggaccactttgtacaagaaagctgggtctcagtggtggtggtggtg